(19) United States
(12) Patent Application Publication
(10) Pub. No.: US 2018/0256174 A1
(43) Pub. Date: Sep. 13, 2018

(54) DIAMOND TIP BUR

(71) Applicants: Bryan DEENY, Belleek (IE); Patrick CUSHEN, Mount Olive (IE); Damian CURTIN, Tralee Co (IE); James O'CONNOR, Bishopsown (IE)

(72) Inventors: Bryan DEENY, Belleek (IE); Patrick CUSHEN, Mount Olive (IE); Damian CURTIN, Tralee Co (IE); James O'CONNOR, Bishopsown (IE)

(21) Appl. No.: 15/897,714

(22) Filed: Feb. 15, 2018

Related U.S. Application Data
(60) Provisional application No. 62/468,450, filed on Mar. 8, 2017.

(51) Int. Cl. A61B 17/16; B23B 27/20

(52) U.S. Cl. CPC A61B 17/1615; A61B 2017/00238; B23B 27/20

(57) ABSTRACT

An arthroscopic surgical tool arrangement for performing endoscopic surgical procedures which includes a powered handpiece and a cutting accessory which detachably connects to the handpiece is provided. The cutting accessory has a distal end which defines a cutting head incorporating diamond-grit particles.

### CROSS REFERENCE TO RELATED APPLICATION

This claims the benefit of U.S. Provisional Application No. 62/468,450, filed Mar. 8, 2017, the disclosure of which is hereby incorporated by reference in its entirety.

### FIELD OF THE INVENTION

This invention generally relates to a surgical tool arrangement useful for performing endoscopic surgical procedures which includes a powered handpiece and, more particularly, to a cutting accessory which detachably connects to the handpiece and incorporates a diamond-grit bur.

### BACKGROUND OF THE INVENTION

Endoscopic surgical procedures are routinely performed in order to accomplish various surgical tasks. In such a surgical procedure, small incisions or portals are made in the patient. An endoscope, which is a device that allows medical personnel to view the surgical site, is inserted in one of the portals. Surgical instruments used to perform other tasks are inserted into other portals. The surgeon views the surgical site through the endoscope to determine how to manipulate the surgical instruments in order to accomplish the desired procedure. An advantage of performing endoscopic surgery is that, since the portions of the body that are cut open are minimized, the portions of the body that need to heal after the surgery are likewise reduced. Moreover, during an endoscopic surgical procedure, only relatively small portions of the patient's internal organs and tissue are exposed to the open environment. This minimal opening of the patient's body lessens the extent to which the organs and tissue are open to infection.

The ability to perform endoscopic surgery is enhanced by the development of powered surgical tools especially designed to perform such procedures. Once such tool is sold by the assignee hereof under the trademark FORMULA®. This tool is in the form of a cylindrical handpiece designed to be held in the hand of the surgeon. The handpiece has a front or distal end provided with a coupling assembly for releasably holding a cutting accessory, and a motor disposed within a handpiece housing which drives the accessory. The cutting accessories, such as shavers, drills and burs, include a hub which defines the proximal end of the accessory and is appropriately configured to cooperate with the coupling assembly of the handpiece to lock the accessory thereto, an elongated and tubular housing element having a proximal end fixed to the hub, and an elongated cutting element including a drive shaft disposed within the housing element. When the accessory is attached to the handpiece, the handpiece motor couples to the drive shaft of the accessory and moves same relative to the outer housing element. The handpiece motor is selectively actuable to drive the accessory drive shaft so as to cause a desired cutting action at the distal end of the accessory. The handpiece is associated with a control unit which controls the functioning thereof, and is actuated by the user via appropriate buttons provided on the handpiece itself, at the control unit or through use of a footswitch.

In an endoscopic surgical procedure, irrigating fluid is introduced into the surgical site. This fluid serves as a transport media for removing tissue and debris from the surgical site. In order to remove the irrigating fluid and the material contained therein, the above handpiece and the various accessories which are usable therewith together define a suction conduit. A suction pump is connected to the handpiece to provide the suction force needed for drawing the fluid and material away from the surgical site. In order to control the suction flow through the accessory and the handpiece, the handpiece is typically provided with a manually operated valve which is manipulated by the surgeon to control suction of material away from the surgical site.

Mechanical cutting accessories, such as the shaver, drill and bur discussed above, are commonly used in arthroscopic procedures, and allow for the resection of hard and soft bodily tissues, for example, those found within the knee, shoulder and other joints. A bur-type cutting accessory is commonly used to resect bone or other hard tissues, and includes cutting features which when rotated serve to cut away targeted bone or hard tissue. The cutting element of a bur-type cutting accessory includes a cutting head with these cutting features which are exposed through a window formed at the distal end of the outer housing element when the cutting element is located therein. In some bur-type cutting accessories, the window formed in the outer housing element opens primarily sidewardly, so that the distal end of the outer housing element covers a portion or one side of the cutting head of the bur to allow the user to better target bone or hard tissue. Alternatively, the entire cutting head geometry may project distally beyond the terminal end of the outer housing element, and this type of bur configuration is often called “unhooded”. Many bur configurations are for removal of particular bone or hard tissue types, and a variety of different bur geometries are available to specifically address the type of cutting the accessory is to carry out.

Further, in some conventional bur-type cutting accessories, the cutting element includes an elongate and hollow tubular drive shaft and the cutting head at the distal end thereof is provided as a solid member which is fixedly mounted to the distal end of the drive shaft. In order to draw suction through the cutting element in this type of accessory, the distal end of the drive shaft is provided with a suction opening which opens sidewardly outwardly and communicates with the hollow interior of the drive shaft. In operation, bone or other hard tissue removed or cut away by the cutting head is suctioned into the hollow interior of the drive shaft through the window of the outer housing element. Bur-type cutting accessories, due to their configuration, often spray surgical debris outwardly and away from the cutting features of the cutting head. The surgical debris will be evacuated from the surgical site through the suction opening located proximally from the cutting head of the bur.

While the arrangements described above serve to remove fluid and surgical debris from the surgical site, there is a continuing desire and need for improved performance in surgical tools in an effort to minimize trauma to the patient and to make the operative procedure more efficient and effective.

### BRIEF DESCRIPTION OF THE DRAWINGS

FIG. 1 is a perspective view of an embodiment of the surgical tool arrangement according to the invention, including a handpiece with a surgical accessory attached thereto;

FIG. 2 is an enlarged, fragmentary, longitudinal and cross-sectional view of the handpiece of FIG. 1 with a surgical accessory attached thereto;

FIG. 3 is an enlarged top and fragmentary view of the surgical accessory;

FIG. 4 is an enlarged longitudinal cross-sectional view of the surgical accessory of FIG. 3, as seen generally along line IV-IV in FIG. 3;

FIG. 5 is an enlarged side view of the tip of the cutting head of the surgical accessory; and

FIG. 6 is an enlarged cross-sectional view of the tip of the cutting head of FIG. 5.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the arrangement and designated parts thereof. The words “forwardly” and “distally” will refer to the direction toward the end of the arrangement which is closest to the patient, and the words “rearwardly” and “proximally” will refer to the direction toward the end of the arrangement which is furthest from the patient. Said terminology will include the words specifically mentioned, derivatives thereof, and words of similar import.

### DETAILED DESCRIPTION

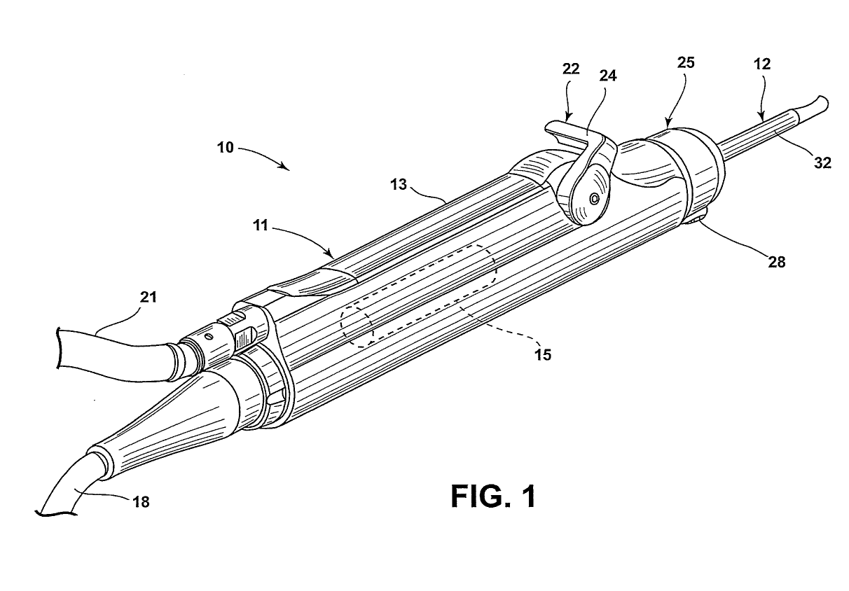

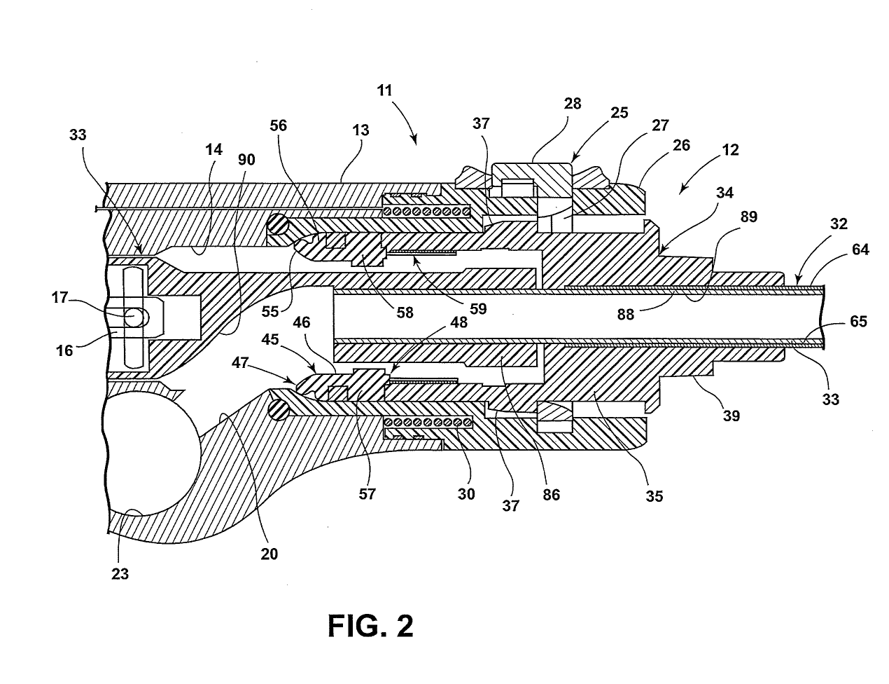

Referring to FIGS. 1 and 2, a surgical tool arrangement 10 according to the invention is illustrated. The arrangement 10 includes a handpiece 11, which at its distal end mounts thereon a surgical accessory 12.

Handpiece 11 is a commercially available surgical handpiece manufactured by the assignee hereof, under Model Nos. 375-704-500 and 375-701-500, and is accordingly only briefly described herein. Handpiece 11 includes an elongate outer housing 13 defining an elongate bore 14 therein. A motor 15 (shown diagrammatically only in FIG. 1) is disposed within housing bore 14. Motor 15 includes an output or drive shaft 16, which drive shaft 16 mounts a drive pin 17 at the distal end thereof. A power cable 18 is coupled to the proximal end of handpiece 11 for supplying power to motor 15.

Handpiece housing 13 defines therein an elongate suction bore (not shown) extending generally parallel to and sidewardly of housing bore 14. This suction bore communicates with a diagonally extending suction passage 20 defined in housing 13, which passage 20 provides communication between the distal end of housing bore 14 and the suction bore. Suction is drawn through the handpiece 11 by a suction pump (not shown), which is connected to the handpiece 11 via a suction tube 21. Suction flow through the handpiece 11 is regulated by an adjustable valve 22 having a valve stem (not shown) which is movably mounted in a valve bore 23 defined in housing 13. The valve 22 is adjusted by the user via a movable handle or arm 24 connected to the valve stem. The above handpiece suction arrangement is described in detail in U.S. Pat. No. 7,682,333 issued on Mar. 23, 2010, which patent is owned by the same assignee hereof and is hereby incorporated by reference herein in its entirety.

The accessory 12 is removably attached to the distal end of the handpiece 11 by a coupling assembly 25 provided on the handpiece 11. Coupling assembly 25 includes a generally ring-shaped collet 26 secured to the distal end of the handpiece housing 13. A locking ring 27 is movably disposed in collet 26 and is biased to hold the accessory 12 within the housing bore 14 of handpiece 11. A release button 28 is provided on locking ring 27, and is used to release the locking ring 27 and allow removal of the accessory 12 from handpiece 11. Further, a coil 30 is provided in collet 26, which is used to facilitate inductive signal transfer to/from a radio-frequency identification device (RFID) disposed in the accessory 12 as discussed below.

Referring to FIGS. 2-4, the accessory 12 will now be described. Accessory 12 defines a central longitudinal axis 31, and includes an outer cannula or tubular housing element 32 and a tubular cutting element 33 disposed within housing element 32. Housing element 32 includes a hub 34 which defines the proximal end thereof. Hub 34 is defined by a generally tubular base body 35, which defines therein a pair of generally rectangular and diametrically-opposed openings 36 adjacent the proximal end thereof. Base body 35 also has formed thereon a pair of outwardly-projecting, diametrically opposed and generally ramp-shaped ears 37 disposed distally of openings 36. Ears 37 cooperate with coupling assembly 25 of handpiece 11 to secure accessory 12 therein. Hub 34 has a distal end defined by a head 39 or nose of a reduced diameter as compared to base body 35. Further, hub 34 defines therein a bore 41 which extends completely through the hub 34, and with which openings 36 of base body 35 communicate.

An annular seal 45 is disposed within the proximal end of bore 41 of hub 34. Seal 45 is constructed of a resilient elastomeric material, and is defined by a main section 46 and axially-spaced proximal and distal sections 47 and 48 disposed at respective opposite ends of the main section 46. Proximal section 47 defines thereon a pair of annular ribs 55 and 56, which are disposed in sealing engagement with an inner annular surface of collet 26 of handpiece 11 when accessory 12 is coupled thereto, as shown in FIG. 2. Distal section 48 defines thereon a pair of outwardly projecting and diametrically-opposed lock tabs 57 which engage within the respective openings 36 of hub 34 to secure the seal 45 to hub 34 and fix the axial position of seal 45 relative thereto. Distal section 48 additionally defines thereon a pair of inwardly projecting and diametrically-opposed stop tabs 58, which are generally radially aligned with the respective lock tabs 57. As shown in FIGS. 2 and 4, an RFID device 59 encapsulated within a ring structure is located within hub bore 41 distally from, and in axially-adjacent relationship with, the distal section 48 of seal 45.

The above-described coupling arrangement of handpiece 11 and the arrangement of the encapsulated RFID device 59 and coil 30 are disclosed in U.S. Pat. No. 7 887 559 issued on Feb. 15, 2011, which patent is owned by the same assignee hereof and is hereby incorporated by reference herein in its entirety.

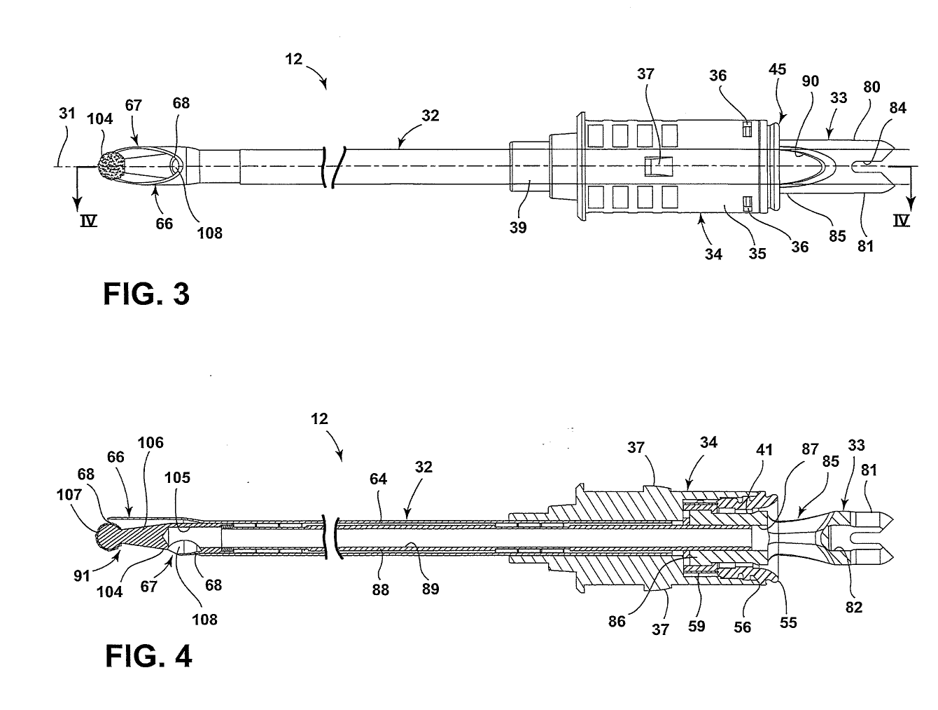

Housing element 32 additionally includes an elongate housing tube 64 which projects distally from hub 34. More specifically, housing tube 64 has a proximal end which is fixedly mounted within the distal portion of bore 41 of hub 34. Housing tube 64 defines an elongate bore or conduit 65 therein, in which the cutting element 33 is disposed as discussed below. Referring to FIGS. 3 and 4, housing tube 64 has a distal end 66 which in the illustrated embodiment is cut so as to define a window 67 having an annular edge 68, which window 67 in the illustrated embodiment opens both sidewardly and distally of the tube 64. Alternatively, the distal end 66 of housing tube 64 may be cut in a manner such that annular edge 68 is oriented perpendicular to the axis 31. Other configurations of distal end 66 of housing tube 64 are within the scope of the invention, and the above are given only by way of example.

Turning now to cutting element 33, same includes a hub 80 which defines the proximal end thereof. Hub 80 incorporates a motor-engaging drive element 81 defining a proximally opening bore 82, and a slot 84 which extends transversely to the longitudinal axis of the cutting element 33. Hub 80 additionally includes a neck 85 which projects distally from drive element 81. Neck 85 terminates at a head 86 which has an enlarged outer diameter. In this regard, the outer diameter of head 86 is slightly larger than the inward projection of the respective stop tabs 58 of seal 45. A bore 87 extends through neck 85 and head 86, in which an elongate and tubular drive shaft 88 is fixed. Drive shaft 88 defines therein a suction passage 89 which is in communication with a suction port 90 defined in neck 85, which suction port 90 is in turn in communication with suction passage 20 of handpiece 11.

Drive shaft 88 has a distal end 91 which mounts a cutting head 104 thereon. In the illustrated embodiment, the drive shaft 88 and the cutting head 104 are constructed as separate components which are fixed to one another. In this regard, the drive shaft 88 may be constructed of a rigid plastic and then induction welded to the cutting head 104, which may be constructed of rigid metal, such as stainless steel. Alternatively, the drive shaft 88 and the cutting head 104 may be constructed as an integral or one-piece member formed from rigid metal, such as stainless steel.

The cutting head 104 preferably has a proximal hollow interior portion 105, a solid frusto-conical shaped neck 106, and a solid substantially spherical-shaped tip 107. A suction port 108 is located at the distal portion of the cutting head 104 and leads to the hollow interior 105. The cutting head 104 is preferably one piece, with the base or substrate made of stainless steel, but may be multiple pieces and made of other materials.

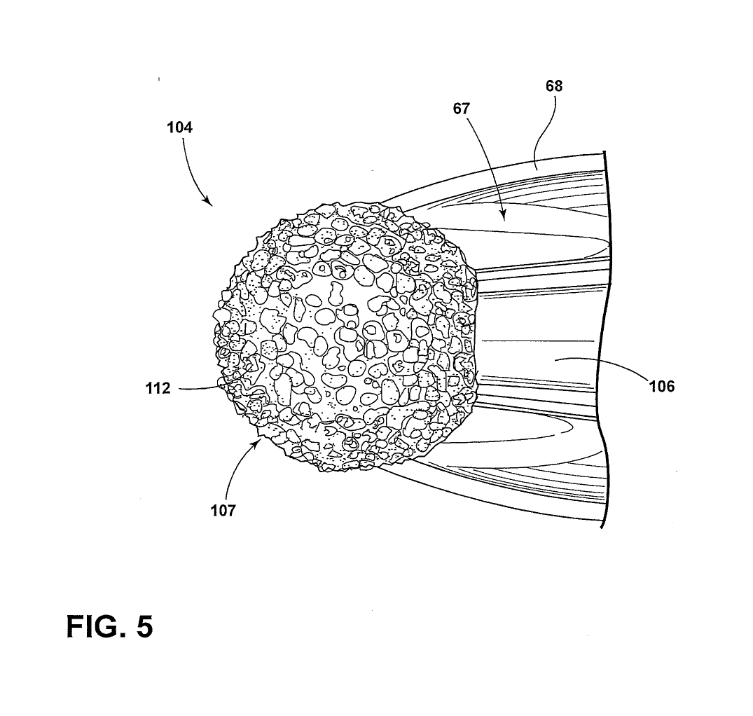

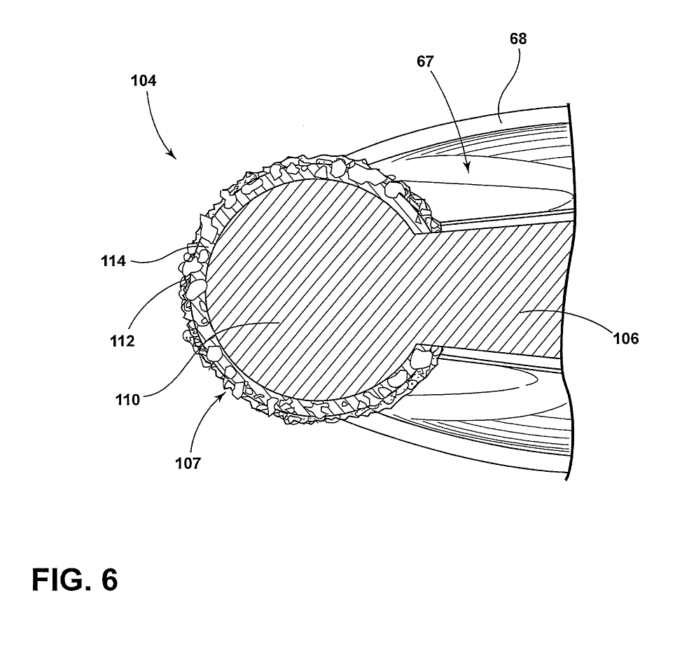

As shown in more detail in FIGS. 5 and 6, the substantially spherical tip 107 has a solid or substantially solid base 110, which, as noted above, is preferably of stainless steel. Attached to the exterior surface of the base 110 are a plurality of diamond-grit particles 112. The diamond-grit particles are preferably synthetic diamond, but may also be of natural diamond. The diamond-grit particles 112 preferably have a nominal diameter of at least 252 microns, and more preferably a nominal diameter 427 microns, with 95% of the particles being less than 455 microns, 93% of the particles being greater than 302 microns, 99.9% of the particles being less than 600 microns, and 99.5% of the particles being greater than 213 microns. It is contemplated that other size grit particles may be used.

The diamond-grit particles 112 are attached to the base 110 by use of a nickel plating 114. The use of the nickel plating 114 results in the diamond-grit particles 112 being bonded to the stainless steel base 110 via co-deposition that mechanically locks the diamonds to the base 110. The nickel plating 114 is electrodeposited onto the base 110, and the diamond-grit particles 112 are preferably coated between about 50 and about 70% of their nominal diameter, more preferably between about 64% and about 68% of their nominal diameter, and most preferably about 66% of their nominal diameter.

The tip 107 is diamond coated with the diamond-grit particles 112 by creating a steel bur blank, which includes the proximal end having hollow interior 105, the neck 106, and the tip 107. The blank is masked so that only the intended cutting surfaces on the tip 107 are coated. The diamond-grit particles 112 and an initial nickel or nickel matrix layer are simultaneously co-deposited onto the blank in a nickel electrodeposition bath. The bath includes a nickel electrolyte solution and containers of diamond-grit particles submerged therein. The steel bur blank is positioned so that the surfaces that require coating are immersed in the layer of diamond-grit particles. A small initial layer of nickel is deposited onto the non-masked exterior surface of the blank via electrodeposition. This layer of nickel builds up around the diamond-grit particles that are touching the blank and mechanically tacks them to the base 110.

After a relatively thin layer of nickel has been built up on the bur head base 110, the part is removed from the electrodeposition bath and placed into another nickel electrodeposition bath that does not contain any diamond particles. The electrodeposition process continues until the nickel layer has reached the desired depth to ensure the diamond-grit particles 112 are securely bonded to the blank.

The tip 107 of the cutting head 104 may be a variety of different diameters as small as 1.0 mm and at least as large as 6.0 mm, but is preferably about 4.0 mm in diameter or about 5.5 mm in diameter. The cutting head 104 is preferably fluteless, but it is contemplated that a tip with both flutes and diamond-grit particles could be provided.

The cutting element 33 is assembled to the outer tubular housing element 32 by inserting the distal end 91 of drive shaft 88 of cutting element 33 into bore 41 at the proximal end of hub 34. During this insertion, the enlarged head 86 of hub 80 expands the seal 45 and the head 86 pushes past the stop tabs 58, at which point the seal 45 essentially resumes its original shape. The stop tabs 58, while allowing some axial displacement of cutting element 33 relative to housing element 32, prevent the cutting element 33 from detaching or falling out of the housing element 32 due to gravitational forces.

The assembled accessory 12 is secured to the handpiece 11 in a similar manner to that described in the '559 patent referenced above, and will accordingly be only briefly described here. Accessory 12 is attached to handpiece 11 by inserting the hubs 34 and 80 into the open distal end of collet 26. The ears 37 of hub 34 seat within collet 26, and the locking ring 27 serves to hold the accessory 12 within handpiece 11. The above securement of the accessory 12 to handpiece 11 causes the drive element 81 to engage the motor output shaft 16. More specifically, the drive pin 17 of output shaft 16 seats within slot 84 of drive element 81, such that the rotational movement of output shaft 16 is transferred to the cutting element 33.

In operation, the distal end of tool 10 is inserted into the surgical site. The cutting element 33 is controlled by a control unit (not shown) connected to handpiece cable 18, which control unit supplies electrical power to the motor 15 of handpiece 11 in order to actuate cutting element 33 and control the rotational speed thereof. If cutting of tissue is desired, then motor 15 is activated so as to cause cutting element 33 to rotate within and relative to outer housing element 32. In this regard, it will be appreciated that the control unit may include appropriate control buttons so as to allow the surgeon or operator to select the desired accessory operations. These control functions of the cutting element 33 may alternatively be performed directly from the handpiece 11 which would then include the appropriate control buttons thereon. Alternatively, the control unit may be associated with a switch, either through a suitable cable or wirelessly, to allow the surgeon to operate the controls remotely. Such a switch may be a footswitch or a hand switch.

As shown in FIG. 4, with the cutting element 33 disposed within housing element 32 and the accessory 12 secured to handpiece 11 as described above, the cutting head 104 is positioned adjacent the window 67 of the housing element 32 so that at least a portion of the cutting head 104 is exposed. In this regard, the cutting head 104 is shown herein as being covered on one side thereof by the distal end 66 of the housing element 32. However, it will be appreciated that other configurations of the housing element distal end 66 are within the scope of the invention. For example, the housing tube 64 of housing element 32 may be provided with a length which allows full exposure of the cutting head 104 axially beyond the distal end 66 of housing tube 64, so that the cutting head is unhooded or uncovered completely.

If desirable or necessary, suction can be provided at the surgical site by manipulating valve 22 on handpiece 11 to draw surgical debris from the surgical site through the suction port 108 and into the interior 105 of the proximal portion of cutting head 104, into drive shaft suction passage 89, into handpiece suction passage 20 and proximally through the handpiece 11 towards the suction pump. In this regard, the positioning of the suction port 108 at the proximal end of the cutting head 104 provides a direct entry port into the interior 105 of cutting head 104. More specifically, when the cutting element 33 is rotating and removing tissue during a surgical procedure and suction is applied, bone or other debris generated by the cutting action of the diamond-grit particles 112 is evacuated from the surgical site via the suction port 108, into the interior 105 of cutting head 104 and on through the handpiece 11.

The use of diamond-grit particles for arthroscopy in particular results in a cutting device with much less “skip” with respect to fluted burs of the same general shape. In some cases, the skip distance was reduced by as much as 71% when using a diamond-grit cutting head versus a fluted head. The use of diamond-grit particles accordingly results in less chatter during an arthroscopic surgical procedure. Thus, the burs of the type described herein increase the control a user has while debriding bone in arthroscopic surgeries.

Although particular preferred embodiments of the invention are disclosed in detail for illustrative purposes, it will be recognized that variations or modifications of the disclosed apparatus, including the rearrangement of parts, lie within the scope of the present invention.

What is claimed is:

1. An arthroscopic surgical device comprising:
an elongated housing tube having a housing proximal end, a housing distal end, and a hollow portion;
an elongated inner shaft having a shaft proximal end and a shaft distal end, the inner shaft disposed at least partially within the housing tube and rotatable with respect to the housing tube; and
a cutting head attached to the shaft distal end and having a tip including a base with a non-flat surface and having diamond-grit particles bonded thereto, the tip being at least partially exposed from the housing tube, the diamond-grit particles being bonded to the surface of the base by a bonding material via electrodeposition, where the diamond-grit particles are between about 50% and about 70% covered by the bonding material.

2. The surgical device of claim 1, wherein the surface of the tip is substantially spherical.

3. The surgical device of claim 2, wherein the diamond-grit particles are about 66% covered by the bonding material.

4. The surgical device of claim 3, wherein the tip has a frusto-conical shaped neck.

5. The surgical device of claim 1, wherein the diamond grit comprises particles having an average nominal diameter of about 427 microns.

6. The surgical device of claim 5, wherein 95% of the diamond-grit particles have a nominal diameter less than 455 microns.

7. The surgical device of claim 1, wherein the bonding material is a nickel matrix.

8. The surgical device of claim 1, wherein the tip has a diameter of about 4.0 mm or about 5.5 mm.

9. The surgical device of claim 1, wherein the diamond grit is comprised of synthetic diamond.

10. The surgical device of claim 9, wherein the diamond grit consists essentially of synthetic diamond.

11. An arthroscopic surgical device comprising:
a handle; and
a disposable arthroscopic cutter removably attached to the handle and comprising:
an elongated housing tube having a housing proximal end, a housing distal end, a hollow interior defined by an outer wall, and a housing window in the outer wall and located adjacent the housing distal end;
an elongated inner shaft disposed at least partially in the hollow interior of the housing tube, the inner shaft having a longitudinal axis, a shaft proximal end and a shaft distal end, the inner shaft being rotatable with respect to the housing tube about the longitudinal axis; and
a bur head attached to the shaft distal end, the bur head comprising a tip that has a rounded portion and an unfluted surface with diamond-grit particles bonded thereto by the use of nickel, the tip being at least partially exposed from the housing tube via the housing window, the diamond-grit particles having an average nominal diameter of at least 252 microns.

12. The surgical device of claim 11, wherein the tip comprises a substantially spherical portion.

13. The surgical device of claim 11, wherein the tip has a base comprised of stainless steel.

14. The surgical device of claim 11, wherein the diamond-grit particles are comprised of synthetic diamond.

15. The surgical device of claim 14, wherein the diamond-grit particles consist essentially of synthetic diamond.

16. The surgical device of claim 11, wherein the diamond-grit particles have a nominal average diameter of about 427 microns.

17. A method of using a surgical device, the method comprising the steps of:
providing a surgical device comprising:
an elongated housing tube having a housing proximal end, a housing distal end, and a hollow portion;
an elongated inner shaft having a shaft proximal end and a shaft distal end, the inner shaft disposed at least partially within the housing tube and rotatable with respect to the housing tube; and
a bur head attached to the distal end of the inner shaft and having a tip with a base having a non-fluted surface and diamond-grit particles bonded thereto, the tip being at least partially exposed from the housing tube, the diamond-grit particles being bonded to the base by a bonding material that covers between about 64% and about 68% of the surface area of the diamond-grit particles;
activating the surgical device such that the inner shaft rotates relative to the housing tube; and
engaging the tip of the bur head to an item to resect a portion of the item.

18. The method of using a surgical device of claim 17, wherein the diamond-grit particles have a nominal average diameter of about 427 microns.
